(19) United States
(12) Patent Application Publication
(10) Pub. No.: US 2004/0115677 A1
(43) Pub. Date: Jun. 17, 2004

(54) Robot-friendly pcr plates

(76) Inventors: Masato Mitsuhashi, Irvine, CA (US); Takayuki Saito, Ibaraki (JP); Hiromasa Kawai, Ibaraki (JP); Chikashi Takase, Ibaraki (JP)

Correspondence Address: KNOBBE MARTENS OLSON & BEAR LLP, 2040 MAIN STREET, FOURTEENTH FLOOR, IRVINE, CA, 92614, US

(21) Appl. No.: 10/467,383

(22) PCT Filed: Jan. 24, 2002
(86) PCT No.: PCT/US02/02260

(51) Int. Cl. C12M 1/34; C12Q 1/68

(52) U.S. Cl. 435/6; 435/91.2; 435/287.2; 435/288.4

(57) ABSTRACT

The present invention relates to microtiter plates for use in automated systems. Specifically, the present invention relates to microtiter plates which are thermally stable under conditions required for polymerase chain reaction (PCR) experiments, while also maintaining the rigidity and dimensions necessary for mechanical manipulation in automated systems.

### FIELD OF THE INVENTION

The present invention relates to microtiter plates, specifically plates designed for polymerase chain reaction (PCR) applications, for use in automated systems.

### DESCRIPTION OF THE RELATED ART

Polymerase chain reaction (PCR) is a widely used in vitro biochemical protocol. PCR has proven to be a phenomenal tool for diagnostics and research in many scientific fields including genetics, molecular biology, cellular biology, clinical chemistry, forensic science, and analytical biochemistry. Reactions which are performed by carefully controlling and cycling the reaction temperature during PCR, include, but are not limited to, chemical amplification of nucleic acid sequences, ligase chain reaction, and nucleic acid sequencing. PCR is typically carried out in small tubes or in multi-well microtiter plates which are placed in contact with a thermal cycler. Researchers use microtiter plates to conduct potentially hundreds of PCR experiments relatively efficiently and in very low volumes. The addition and removal of PCR reagents and products from many wells in very small, specific volumes is preferably done mechanically, specifically by automated computer-programmed robotics.

Automated systems are being used in the field of biochemistry for high throughput screening of samples. Automated detection systems may include fluorimagers and other biochemical detection systems. These systems may be machines including, but not limited to, Applied Biosystem's ABI PRISM® 7900 HT Sequence Detection System, Northstar® HTS Workstation, COBRA™ Robotic Sample Handling System, and FMAT ™ 8100 HTS System. Automated systems may, for example, use a robotic arm to handle articles, such as microtiter plates, used in various screening protocols. These articles are typically made of a hard polystyrene material, which allows the robotic arm to easily grasp the article. However, polystyrene does not exhibit thermostability, so polypropylene is used in articles which must be heated, for example during PCR experiments. Polypropylene is not suited for use in automated systems, however, because it is soft and tends to deform when handled.

Microtiter plates for use in PCR applications are made of heat-stable plastic materials, such as polypropylene, polyethylene, and polycarbonate. Unlike robot-compatible polystyrene, which provides enough rigidity for robots to grasp, transfer to exact locations, or stack multiple plates together, heat-stable plastic materials lack such rigidity and manipulability. Additionally, polypropylene is a partially opaque material, i.e., it appears cloudy or not fully transparent. Thus, it is difficult to see samples once they are in the wells. Also, plates consisting of heat-stable materials are not suitable for fluorescent end point measurement and real time PCR, as the material causes a change in the light path.

PCR requires multiple cycles of 94 to 95° C. treatment in which plates made of polypropylene often deform, for instance by shrinking, twisting, and warping. This change in dimensions of the microtiter plate causes a critical problem. Heat conductivity among multiple wells of the microtiter plate is not uniform on a deformed plate, because the dimension of heat applied to the plate is fixed and is not designed to conform to the altered shape of the microtiter plate. Additionally, if microtiter plates which are not thermally stable are manipulated in an automated liquid handler following PCR, the change in dimensions caused by twisting or warping of the plate is problematic. In this situation, dispenser nozzles may have difficulty reaching the bottom of each well of the plate. In some cases, the dispenser nozzle may touch the side of the well and never reach the bottom of the well. If the dispenser nozzle is prevented from contacting the bottom of the well, a bubble may form in the well when the solution is dispensed into the well, or alternatively a substantial volume of the solution may be left in the well when the solution is removed from the microtiter plate. This is of major concern to researchers who wish to use automated systems in the preparation and execution of PCR experiments. Inaccuracies in dispensing solutions into wells and in removing products following PCR can cause significant errors in measurements, such as concentration, and detection of molecules of interest.

Furthermore, when sealing caps are applied to the wells of a deformed microtiter plate, solutions in the wells may evaporate and leak from the space between the sealing cap and the plate during PCR Moreover, once the dimension of the plate is changed following PCR, a robot can not effectively manipulate and remove the plate from the thermal cycler. Automated systems are programmed electronically to locate a plate and individual wells according to specific parameters, so the machine will not he able to identify changes in dimension or location of the plate and wells should they become deformed. Therefore, changes in the location of wells over the course of a PCR experiment caused by deformation of the microtiter plate is not conducive to the use of robotics in automated systems. The problems caused by deformation of the plate during PCR becomes particularly critical in the use of 384 and 1536 well plates, as smaller wells and liquid volumes are used.

Increasing the thickness of the plastic materials used may prevent deformation to a certain extent, but a key factor in PCR experiments is heat conductivity. In order to maintain rapid heat conductivity from the thermal cycler through the wall of a PCR plate well the wall thickness should be thin, typically about 0.3 m.

### SUMMARY OF THE INVENTION

The present invention relates to a microtiter plate for use in polymerase chain reaction applications which is made of a composition comprising syndiotactic polystyrene. The microtiter plate of the present invention may be a 96, 384, or 1536-well plate.

The present invention also relates to a microtiter plate for use in polymerase chain reaction applications which is made of a composition comprising cyclic olefin polymer. The cyclic olefin plate may be a 96, 384, or 1536-well plate.

The present invention further relates to a microtiter plate made of a composition comprising cyclic olefin polymer, wherein the polymer is produced by addition polymerization, addition copolymerization, ring opening metathesis polymerization, or ring opening metathesis copolymerization of monomers of norborene. The present invention also relates to a microtiter plate made of a composition comprising cyclic olefin polymer, wherein the polymer is produced by addition polymerization, addition copolymerization, ring opening metathesis polymerization, or ring opening metathesis copolymerization of monomers of a cyclopentadiene.

The present invention also relates to a microtiter plate for use in polymerase chain reaction applications consisting essentially of either syndiotactic polystyrene or cyclic olefin polymer.

### BRIEF DESCRIPTION OF THE DRAWINGS

FIG. 1 is a graphical representation of the end-point fluorescence intensity measured following PCR performed in an ABI 384-well PCR plate and a 384-well plate of the present invention which was pre-annealed. Intensity is shown in relation to -actin plasmid DNA concentration. The data is further described in Example 1.

FIG. 2 is a graphical representation of the change in fluorescence measured at each cycle of PCR performed on annealed 384-well plates of the present invention, non-treated 384-well plates of the present invention, ABI 384-well plates, and ABgene® Thermo-fast 384-well plates, respectively. Fluorescence was measured at each of the 40 PCR cycles. The data and PCR conditions are further described in Example 1.

FIG. 3 is a graphical representation of the change in dimension of a PCR plate of the present invention. Changes in width, length, height, and warp were measured following treatment of the plates under various conditions. Changes to the plate of the present invention can be compared to deformation observed in a standard ABI plate.

FIG. 4 illustrates analysis of PCR products by electrophoresis in an agarose gel. PCR experiments were carried out under the same conditions, but on four different PCR plates. Details of the PCR conditions and plates are described in Example 3.

### DETAILED DESCRIPTION OF THE PREFERRED EMBODIMENT

The ideal plastic material for use in automated PCR applications exhibits the following characteristics:

1. Rigidity at least similar to that of polystyrene;

2. Heat stability at 94-95° C.;

3. Minimal deformity (shrink, twist, warp, etc.) after 94-95° C. treatment under humid conditions;

4. Mass production capability by injection molding technology;

5. Fast heat conductivity, at least similar to polypropylene;

6. Minimal non-specific absorption of protein, DNA, RNA, oligonucleotides, and nucleotides; and

7. Minimal or at least predictable shrinkage factor for injection molding.

Syndiotactic polystyrene and cyclic olefin polymers exhibit thermostability, have the same hardness as polystyrene, and both are suitable for injection molding. Additionally, the inventors of the present invention have found through experimentation that no adhesion of non-specific DNA or protein is observed with articles made from these materials, specifically PCR plates. The inventors have created microtiter plates made primarily of either syndiotactic polystyrene and cyclic olefin polymers which are capable of maintaining thermal stability and rigidity sufficient to meet standards required for PCR and manipulation in automated systems.

Cyclic olefin polymer may be obtained from Nippon Xeon (tradenames Zeonex® and Zeonor®), Mitsui Chemical (tradename Apel), and JSR Corporation (tradename Arton®). Microtiter plates of the present invention may be constructed of various grades of cyclic olefin, for instance, 1410R grade cyclic olefin (Nippon Zeon, Japan). Cyclic olefin polymers, also referred to as cyclo-olefin polymers, are engineered thermoplastics derived from the ring-shaped norborene molecule, which is made from dicyclopentadiene (DCPD) and ethylene. Alicyclic olefin resin may be produced by addition polymerization, addition copolymerization, ring opening metathesis polymerization, or ring opening metathesis copolymerization of monomers. The resin exhibits thermal stability, low moisture absorption, dimensional stability, and high melt-flow rates. Specifically, Zeonex® and Zeonor® also exhibit chemical resistance and very low fluorescence. The cyclic olefin microtiter plates of the present invention are thermally stable up to 140° C.

Syndiotactic polystyrene (SPS) is made by metallocene catalysis polymerization. Syndiotactic polystyrene, under the trade name Xarec®, may be obtained from Idemitsu Petrochemical Company (Tokyo, Japan). Microtiter plates of the present invention may be constructed of various grades of SPS, for instance S100 grade. The syndiotactic polystyrene microtiter plates of the present invention are stable up to 250° C.

The microtiter plates of the present invention are formed preferably by the common and well-known method of injection molding of the plastic materials, but may be formed by other means known by those of skill in the art. The composition of the microtiter plates may contain polymers, colorants, lubricants, or other additives known to those of skill in the art.

Wells of a microtiter plate can be arranged in a strip or in-line format, or can be arranged in a matrix format. One microtiter plate configuration has 8×12 wells spaced at 9 mm apart between the wells' centers, for a total of 96 wells. For high throughput screening, other configurations may be created by increasing the total number of wells while keeping the overall size of the well plate the same, for instance a 384-well plate, configured to have 16×24 wells 4 spaced at 4.5 mm apart between the wells' centers and a 1536-well plate configured to have 32×48 wells spaced at 2.25 mm apart between the wells' centers. Since the overall size of these well plates are the same as the 96-well plate, the size of the wells in the 384 and 1536 well plates is necessarily smaller than those in the 96-well plates while the depth of the wells remains the same. The overall dimensions of microtiter plates are set by an international standard determined by the Society for Biomolecular Screening (SBS).

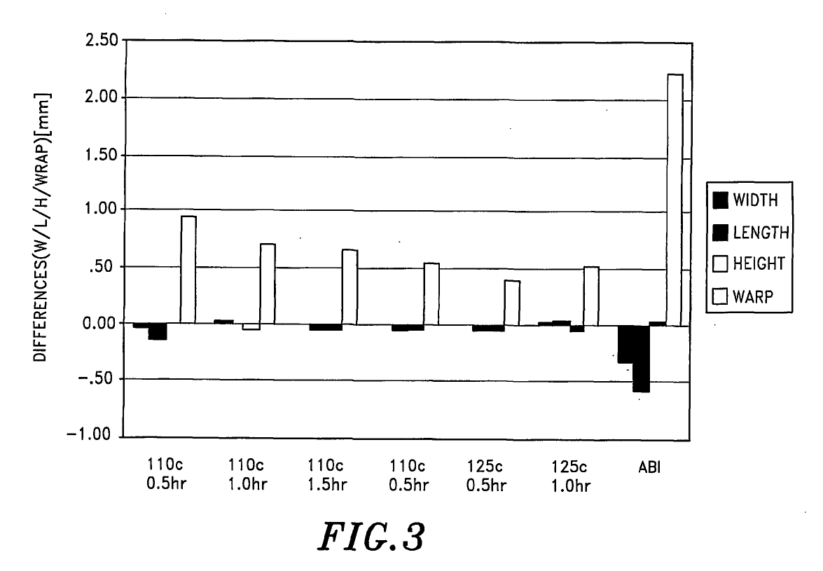

In contrast to microtiter plates presently in use in the field of biochemistry for PCR applications, the microtiter plates of the present invention exhibit little or no warping before, during, or following PCR experiments, as illustrated by FIG. 3 and described in Example 2. This provides a consistent environment in which PCR experiments may be performed, as it allows uniform contact between the microtiter plate and the thermal cycler and the precise dimensions of the plate are maintained to allow for easy manipulation in automated systems. These conditions are ideal for high throughput screening procedures, particularly those in which plates containing many wells, such as 384-well plates, are used.

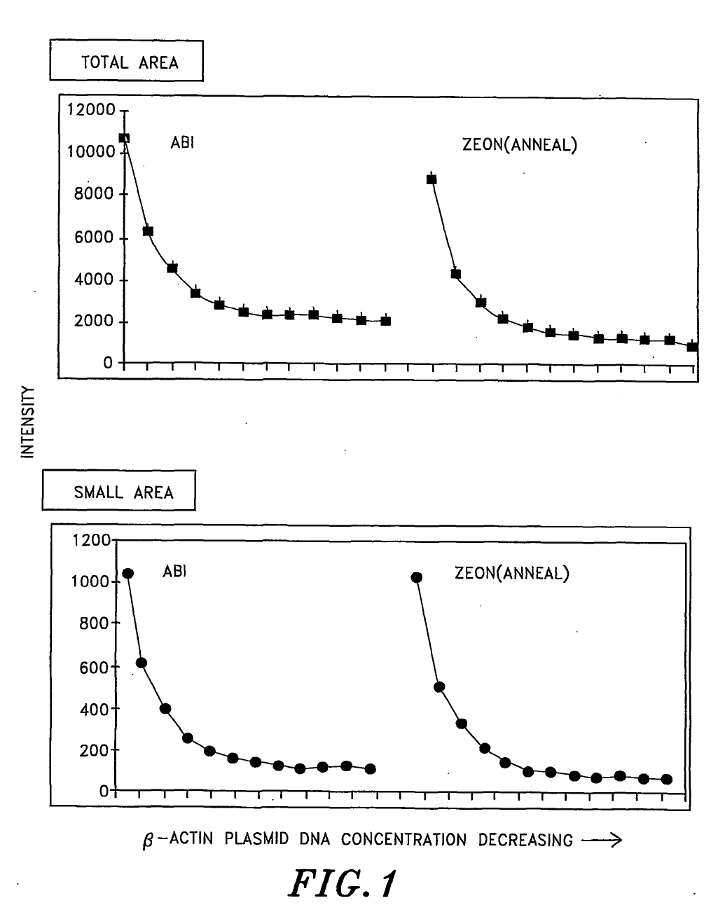

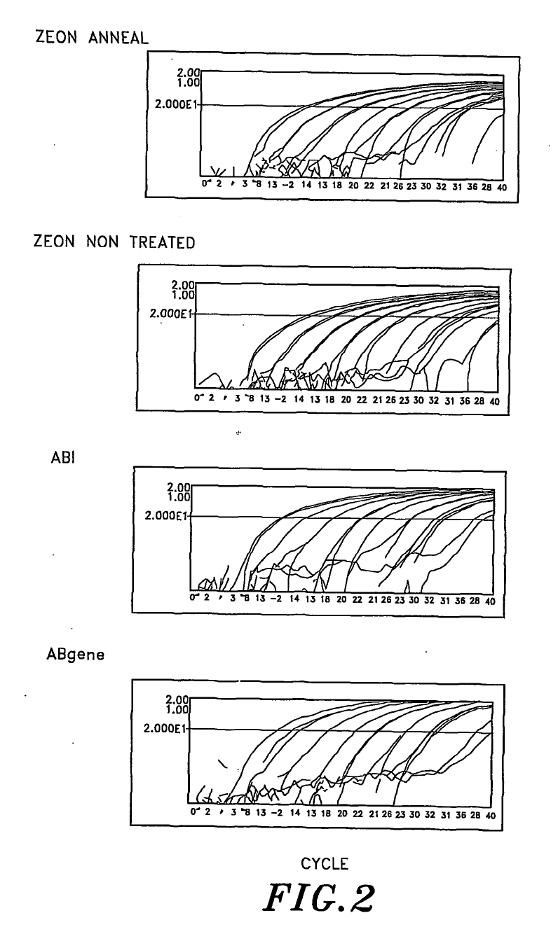

In addition, microtiter plates of the prior art have a cloudy appearance which prevents identification of bubbles which may form at the bottom of wells on the plate. The transparency of the plastic materials used in the present invention allows for easy viewing of solutions in the well of the present microtiter plates. The materials used in the present invention also exhibit very low autofluorescence compared to materials used in the prior art, making the present invention suitable for use in real time PCR applications, as shown in FIGS. 1 and 2 and described in Example 1.

### EXAMPLE 1

384-well microtiter plates composed of cyclic olefin (Zeonor®, Nippon Xeon, Japan) were compared to ABI PRISM® 384-well Clear Optical Reaction Plates (Applied Biosystems) and ABgene® Thermo-fast plates by performing Real Time PCR. Some of the 384-well plates of the present invention were annealed at 110° C. for three hours.

PCR was carried out using ABI TaqMan®-actin with ten-fold dilutions of -actin plasmid DNA using an ABI PRISM® 7900 HT Sequence Detection System (Applied Biosystems). ABI TaqMan® contains -actin primers and dual-labeled fluorogenic hybridization probe, which incorporated with one fluorescent dye. FAM served as a reporter, and its emission spectra was quenched by a second dye, TAMRA. 2.5 microliters (3 M) of -actin forward primer, 2.5 microliters (3 M) of -actin reverse primer, and 2.5 microliters (2 M) of the probe described above were mixed with 12.5 microliters of ABI 2X Master Mix, which contains DNA polymerase, dNTP's, and optimized buffer components.

Various concentrations of plasmid DNA were prepared in 5 microliters of DEPC-treated water ranging from 10 ng/sample to 0.1 fg/sample in 10 fold dilution and with duplicates for each dilution. Five microliters of each serial dilution of plasmid DNA was then mixed with 20 microliters of the TaqMan®-actin PCR mix solution described above.

PCR was carried out as follows: 2 minutes at 50° C., 10 minutes at 95° C., and 40 cycles of 15 seconds at 95° C. and 1 minute at 60° C. Fluorescence data was collected at the 60° C step for each of the samples. This data is shown in FIG. 2. The graphs show the fluorescence measured at each cycle for each of the serial dilutions, in duplicate.

After PCR was conducted in the PRISM® 7900, the end-point PCR products in the ABI plate and the Zeonor pre-annealed plate were scanned by a FMBIO® fluorescent imager (Hitachi) with an excitation wavelength of labeled 488 nm and emission wavelength of 505 nm. The intensity was calculated in two ways. Intensity was first calculated based on a large intensity area, which included a well and the surrounding area of 21.68 mm. Intensity was then calculated by selecting a small intensity area, which was the center of well (1.03 mm). Results of these calculations can be seen in FIG. 1.

### EXAMPLE 2

Six cyclic olefin 384-well microtiter plates of the present invention (Zeonor®) were subjected to the following conditions: 110° C. for 0.5, 1.0, and 1.5 hours and 125° C. for 0.5 and 1.0 hours, respectively. These treated plates were then subjected to 50 PCR cycles of 94° C. for 15 seconds and 60° C. for 60 seconds (thermal cycler lid heated to 105° C.) on an ABI PRISM® 7900 (Applied Biosystems) with 20 microliters of water in each of the 384 wells. The width, length, height, and warp of each plate were measured before and after PCR by a caliper, and FIG. 3 is a graphical representation of changes in these dimensions. For comparison, an ABI 384-well Clear Optical Reaction Plate was also tested under the same PCR conditions.

### EXAMPLE 3

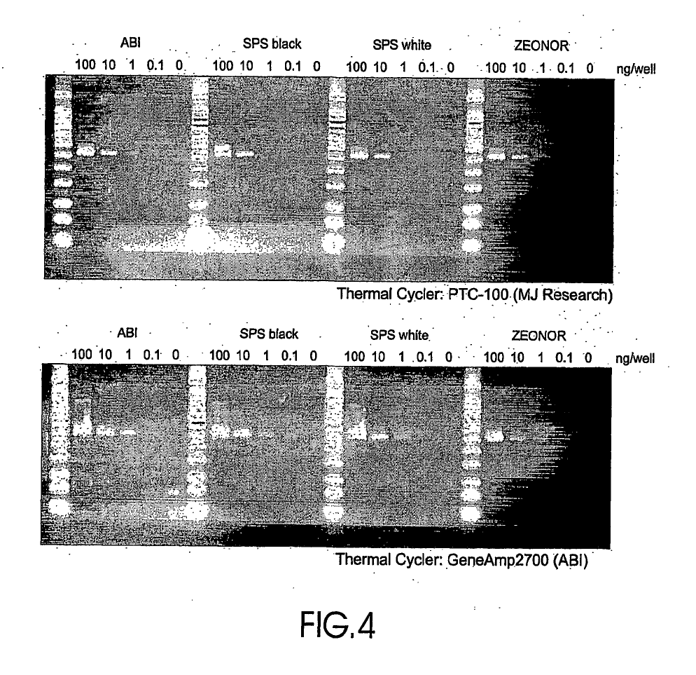

PCR was performed in an ABI MicroAmp® Optical 96-well Reaction Plate, black syndiotactic polystyrene (SPS) 96-well plate of the present invention, white SPS 96-well plate of the present invention, and cyclic olefin (Zeonor®) 96-well plate of the present invention. The PCR reaction mixture contained 0.25 microliters of -actin specific primers, 2.5 mM MgCl₂, 100 mM dNTP, 1×PCR buffer and 1 unit Taq polymerase (Promega). Each individual mixture also included 20 microliters of 100, 10, 1, 0.1, or 0 ng human genomic DNA, respectively. PCR conditions were as follows: 25 cycles of 94° C. for 45 seconds, 60° C. for 1 minute, and 72° C. for 1 minute on either a PTC-100® Thermal Cycler(MJ Research) or a GeneAmp® 2700 thermal cycler (Applied Biosystems). PCR products were analyzed by 2.0% agarose gel electrophoresis, and the results are shown in FIG. 4.

What is claimed is:

1. A microtiter plate for use in polymerase chain reaction applications comprising a plurality of wells, wherein said plate comprises syndiotactic polystyrene.

2. A microtiter plate according to claim 1, wherein said plate is a 96-well plate.

3. A microtiter plate according to claim 1, wherein said plate is a 384-well plate.

4. A microtiter plate according to claim 1, wherein said plate is a 1536-well plate.

5. A microtiter plate for use in polymerase chain reaction applications comprising a plurality of wells, wherein said plate comprises cyclic olefin polymer.

6. A microtiter plate according to claim 5, wherein said plate is a 96-well plate.

7. A microtiter plate according to claim 5, wherein said plate is a 384-well plate.

8. A microtiter plate according to claim 5, wherein said plate is a 1536-well plate.

9. A microtiter plate according to claim 5, wherein said cyclic olefin polymer is produced by a process selected from the group consisting of addition polymerization, addition copolymerization, ring opening metathesis polymerization, and ring opening metathesis copolymerization of monomers of norborene.

10. A microtiter plate according to claim 5, wherein said cyclic olefin polymer is produced by a process selected from the group consisting of addition polymerization, addition copolymerization, ring opening metathesis polymerization, and ring opening metathesis copolymerization of monomers of a cyclopentadiene.

11. A microtiter plate for use in polymerase chain reaction applications comprising a plurality of wells, wherein said plate is constructed of a material that consists essentially of syndiotactic polystyrene.

12. A microtiter plate for use in polymerase chain reaction applications comprising a plurality of wells, wherein said plate is constructed of a material that consists essentially of cyclic olefin polymer.

13. A method for using a plate according to any one of claims 1, 5, 11 or 12, comprising:
placing nucleic acid in at least one of said wells; and
amplifying said nucleic acid in a manner comprising raising and lowering the temperature of said nucleic acid.

14. A method according to claim 13, further comprising detecting the presence of amplified nucleic acid in said well.

15. A method according to claim 14, wherein the detecting comprises quantifying an amount of said amplified nucleic acid.

16. A method according to claim 14, further comprising placing the plate in an automated detection system in which the detecting occurs.
